(19) United States
(12) Patent Application Publication
(10) Pub. No.: US 2012/0116233 A1
(43) Pub. Date: May 10, 2012

(54) SENSOR SYSTEM

(76) Inventor: Robert Weber Mah, Cupertino, CA (US)

(21) Appl. No.: 13/288,800

(22) Filed: Nov. 3, 2011

(30) Foreign Application Priority Data
Nov. 4, 2010 (IN) 3297/CHE/2010

(51) Int. Cl. A61B 6/00; A61B 5/01; A61B 5/103

(52) U.S. Cl. 600/476; 600/587; 600/549

(57) ABSTRACT

A sensor system adapted for characterizing a region of interest within a subject while minimizing bleeding is provided. The sensor system comprises a hollow tool configured to be inserted into the subject, the hollow tool comprising an orifice positioned at an angle with respect to its horizontal axis, a probe disposed within the hollow tool and configured to transmit data from the region of interest; wherein the probe protrudes from the orifice of the hollow tool to be in contact with the region of interest and processing circuitry coupled to the probe and configured to process the sensed data to characterize the region of interest.

### BACKGROUND

The invention relates generally to medical diagnostic systems, and more specifically to a sensor system for tissue characterization.

Traditional methods of diagnosing tissue within the human body have employed invasive techniques such as surgical biopsy procedures to provide accurate diagnosis, and non-invasive techniques such as radiological or magnetic resonance imaging to provide less detailed but useful information as to the condition of tissue. Biopsies involve the removal of tissue from the region of interest where the tissue sample can be subjected to detailed diagnostic to determine the nature and extent of disease.

Percutaneous, image-guided core needle biopsy is being increasingly used to diagnose tissue. Compared to surgical biopsy, this procedure is less invasive, less expensive, faster, minimizes deformity, leaves little or no scarring and requires a shorter time for recovery. However, needle biopsy has a limited sampling accuracy because only a few small pieces of tissue are extracted from random locations in the suspicious mass.

New techniques of tissue characterization include the use of spectroscopy. Specifically, techniques have been developed to use needles carrying optical sensors and percutaneously guided to the region of interest. This enables the sensors to collect enough data to characterize the region of interest. However, the use of needles result in bleeding and which usually hampers tissue diagnosis using optical spectroscopy methods since blood is a strong absorber of light.

Therefore, there is a need to develop a sensor system for tissue characterization that ensures minimum bleeding and accurate data collection to enable reliable characterization of tissue.

### BRIEF DESCRIPTION

Briefly, according to one embodiment of the invention a sensor system adapted for characterizing a region of interest within a subject is provided. The sensor system comprises a hollow tool configured to be inserted into the subject and a probe disposed within the hollow tool and configured to transmit data from the region of interest; wherein the probe protrudes from the orifice of the hollow tool and a tip of the probe is in contact with the region of interest and processing circuitry coupled to the probe and configured to process the sensed data to characterize the region of interest.

In another embodiment, a hollow tool adapted for insertion into a subject is provided. The hollow tool comprises a cylindrical hollow tube with a first end and a second end, and an orifice located adjacent to a first end of the cylindrical hollow tube. The hollow tool further includes a pointed structure attachable to the first end of the cylindrical hollow tube and configured to facilitate entry into the subject and an indexing collar at a second end of the cylindrical hollow tube and configured to enable a rotary motion of the hollow tool when inserted into the subject.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an embodiment of a sensor system implemented according to an aspect of the present technique;

FIGS. 2A, 2B, 2C and 2D are various diagrammatic representations of a hollow tool implemented according to aspects of the present technique;

FIG. 3 is a diagrammatic view of one embodiment of a probe used for obtaining measurements from a region of interest;

FIG. 4 is a diagrammatic view of a hollow tool including a probe disposed therein and implemented according to aspects of the present technique; and

FIG. 5 is a block diagram of an embodiment of a computing device configured to characterize a region of interest according to aspects of the current technique.

### DETAILED DESCRIPTION

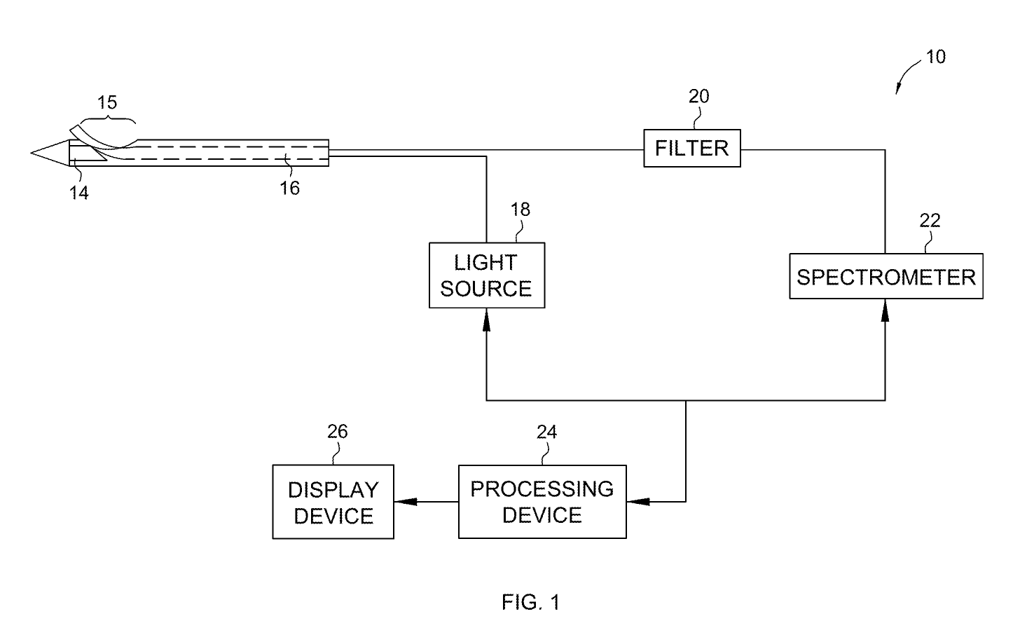

Turning now to the drawings, and referring first to FIG. 1, the present invention will be described as it might be applied in conjunction with a sensor system using optical sensors. In general, however, it should be borne in mind that the present techniques may be used with sensor systems employing other sensors such as thermal sensors and the like. The sensor system is used to characterize a region of interest within subject 12. The illustrated embodiment of the sensor system 10 includes several components and each component is described in further detail below.

Hollow tool 14 is configured to be inserted percutaneously into the subject (not shown) and traverse to a region of interest within the subject. In one embodiment, the hollow tube is a needle. In one embodiment, the hollow tool is adapted to receive a probe 16 within it. In another embodiment, the probe is embedded within the hollow tool and forms a single unit.

Probe 16 is disposed within the hollow tool 14 such that the probe protrudes from the orifice 15 in the hollow tube as shown. When the hollow tube is inserted with the patient, the probe protrudes out of the orifice and is in contact with the region of interest. In one embodiment, the probe is configured to transmit data from the region of interest. In one embodiment, the probe comprises a single optical fiber. In another embodiment, the probe includes multiple optical fibers bundled together. It may be noted that the arrangement of multiple optical fibers can be such that the fibers are packed or are at a distance from each other with supporting spacers disposed there between. In another embodiment, the probe includes optical fiber(s) and other sensor types (e.g. temperature).

Light source 18 provides light of a selected wavelength or band of wavelengths or white light. There may be a filtering device 20. The filtering device 20 is used to precisely select the desired wavelength or band of wavelengths for the procedure. The light is directed to the probe 16 disposed within the hollow tool 14.

While performing the procedure, the hollow tube is inserted into the subject 12 and guided towards a region of interest. The probe 16 disposed within the hollow tool 14 is advanced out the orifice to contact the region of interest. When light enters the region of interest, it absorbs and reflects light through various mechanisms including autofluorescence and scattering.

The optical data reflected by the region of interest is provided to spectrometer 22 as illustrated in FIG. 1. However, it may be noted that other systems suitable for analysis of the radiation from the tissue can also be used. The spectral pattern received by the spectrometer is analyzed by processing circuitry 24.

Processing circuitry coupled to the spectrometer is configured to process the optical spectrum generated by the spectrometer to characterize the region of interest. The region of interest is characterized based on variables such as light intensity patterns, wavelength ratios, look up tables, etc. The results generated by the processing circuitry may be displayed using display device 26. The hollow tool is described in further detail below.

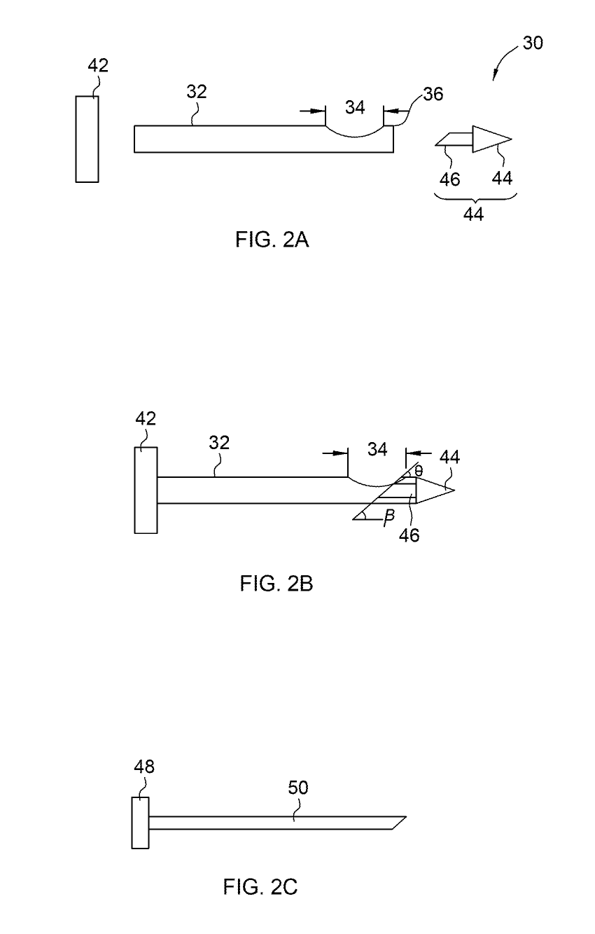

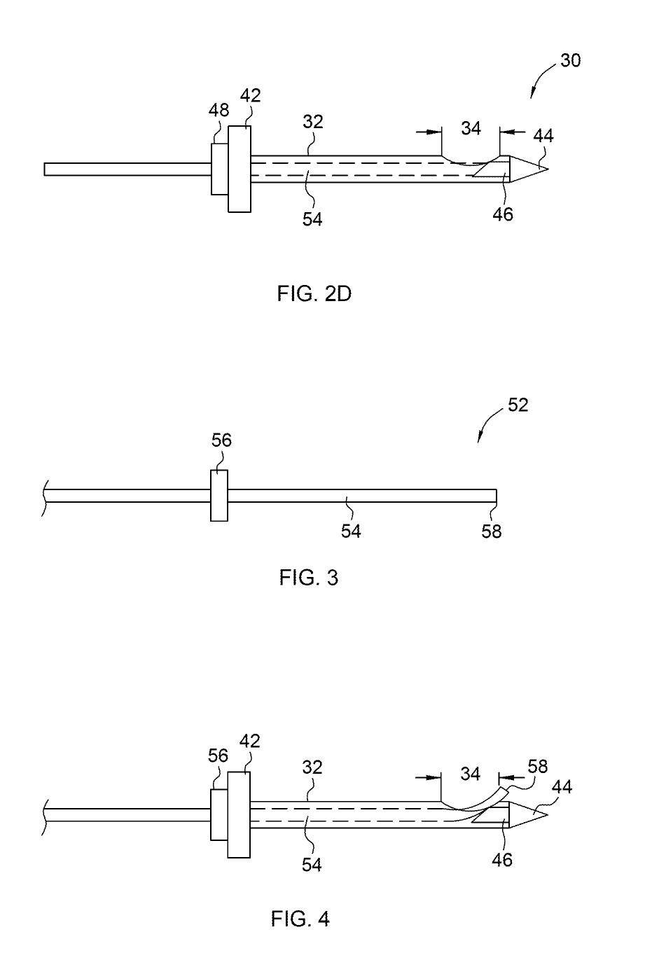

FIG. 2A is an exploded view of a hollow tool and FIG. 2B is an assembled view of a hollow tool used adapted for use with a sensor system implemented according to aspects of the present technique. The various parts of the hollow tool are described with reference to FIG. 2A.

The hollow tool 30 includes a cylindrical hollow tube 32 with a first end 36 and a second end 38. An orifice 34 is formed adjacent to the first end 36. The hollow tool 30 further includes pointed structure 44 that includes a sharp component 44 and guiding component 46 for the probe. The guiding component is formed at an angle “β” with respect to the horizontal axis. The angle “β” can vary from about 10 degrees to 45 degrees. The hollow tool further includes an indexing collar 42.

As shown in FIG. 2B, the indexing collar is disposed on the second end 38 of the cylindrical hollow tube. The indexing collar provides an indication of orientation of the orifice on the hollow tube. In one embodiment, the indexing collar is configured to rotate in increments up to 360 degrees to provide substantial coverage of a region of interest.

FIG. 2C is a diagrammatic view of a stylet used with the hollow tool to reduce bleeding. The stylet 52 includes an indexing collar 48 is disposed on one end of a solid cylindrical tube 50. The stylet is configured to minimize bleeding that normally occurs when the hollow tool is inserted into the subject. The stylet is inserted into the hollow tool 30 as shown in FIG. 2D closing the orifice.

The hollow tool is rotated to a desired degree by the indexing collar. When the hollow tool is positioned at a desired position, the stylet is removed from the hollow tool. A probe is then inserted and required measurements are taken. The manner in which the probe is inserted is described in further detail below.

FIG. 3 is a diagrammatic view of a probe used to transmit and/or receive signal from a region of interest within a subject. The probe 52 includes a sensing component 54 and an indexing collar 56. In one embodiment, the sensing component includes a single optical fiber. In another embodiment, the sensing component includes multiple optical fibers. In such an embodiment, the orientation of the probes at its tip can be the same or can be different based on the measurement requirements. In alternate embodiment, the sensing component includes thermal sensors. The sensing component may be encapsulated with a suitable insulating material to prevent loss of energy.

In the illustrate embodiment, the tip is at a 90 degree angle with respect to a horizontal axis. However, it may be noted that the tip of the probe may also be at angle ranging from 0 to 90 degrees. The manner in which the probe is used along with the hollow tool is described in detail below.

FIG. 4 is a diagrammatic view of the hollow tool carrying a probe adapted for use with a sensor system implemented according to aspects of the present technique. The indexing collar 42 is used to orient the orifice in a desired direction. The probe 52 is inserted into the hollow tool such that portion 58 of the sensing component 54 protrudes from the orifice 34. In one embodiment, a diameter of the orifice 34 is larger than a diameter of the sensing component 54. In general, the orifice is elongated to enable the probe to protrude out the hollow needle to contact tissue.

In one embodiment, the indexing collar 42 of the hollow tool and the indexing collar 56 of the probe are locked with each other using a locking mechanism. The locking mechanism assists in keep the probe and the hollow tool in place while performing a procedure.

The hollow tool is percutaneously guided towards the region of interest and the probe is advanced out the orifice and activated to take measurements. In one embodiment, the hollow tool is graduated to determine the depth of the region of interest. Further, an angle at which the probe contacts the region of interest can also be determined. The hollow tool is capable of being rotated to obtain measurements from areas surrounding the region of interest. In order to rotate the hollow tool, the probe 56 is first removed from the hollow tool and the indexing collar describe din FIG. 2C is inserted in its place.

The hollow tool is then rotated to a desired angle and the indexing collar is removed. The probe is reinserted into the hollow tool and further measurements are taken. These measurements are transmitted to the spectrometer and processing circuitry as described in FIG. 1. Processing circuitry can be implemented on a computing device as described below.

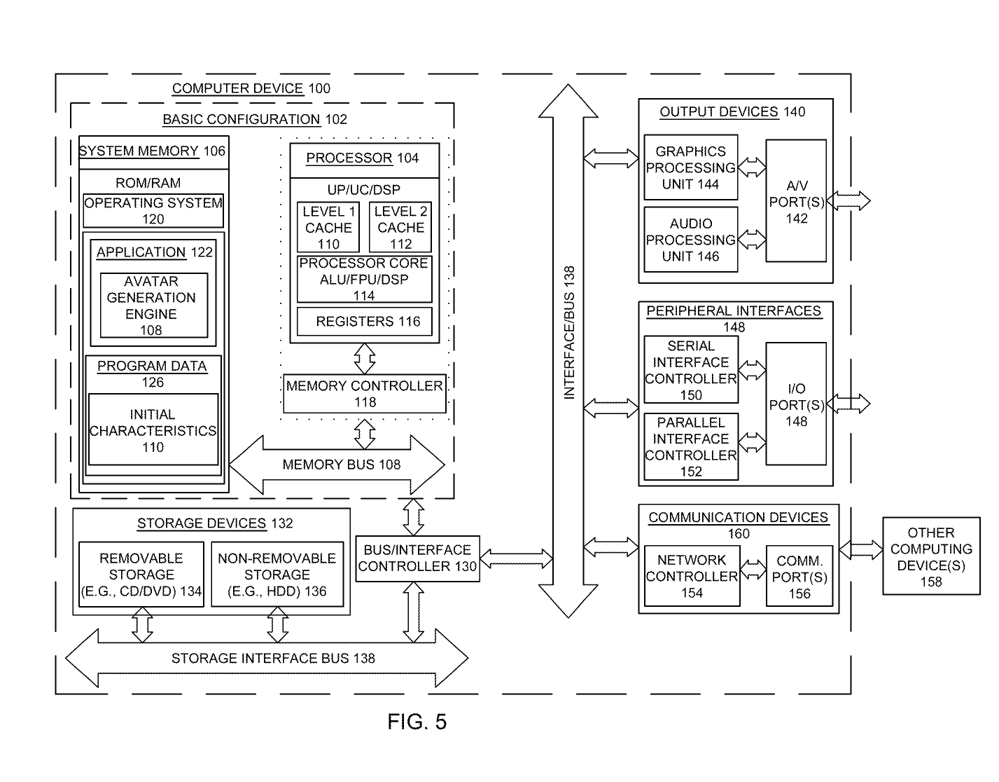

FIG. 5 is a block diagram illustrating an example computing 100 that may be arranged to transmit information regarding a call center in accordance with the present technique. In a very basic configuration 102, computing device 100 typically includes one or more processors and a system memory 106. A memory bus 108 may be used for communicating between processor 104 and system memory 106.

Depending on the desired configuration, processor 104 may be of any type including but not limited to a microprocessor (μP), a microcontroller (μC), a digital signal processor (DSP), or any combination thereof. Processor 104 may include one more levels of caching, such as a level one cache 110 and a level two cache 112, a processor core 114, and registers 116. An example processor core 114 may include an arithmetic logic unit (ALU), a floating point unit (FPU), a digital signal processing core (DSP Core), or any combination thereof. An example memory controller 118 may also be used with processor 104, or in some implementations memory controller 118 may be an internal part of processor 104.

Computing device 100 may have additional features or functionality, and additional interfaces to facilitate communications between basic configuration 102 and any required devices and interfaces. For example, a bus/interface controller 130 may be used to facilitate communications between basic configuration 102 and one or more data storage devices 132 via a storage interface bus 138. Data storage devices 132 may be removable storage devices 134, non-removable storage devices 138, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 106, removable storage devices 134 and non-removable storage devices 136 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 100. Any such computer storage media may be part of computing device 50.

Computing device 100 may also include an interface bus 138 for facilitating communication from various interface devices (e.g., output devices 140, peripheral interfaces 148, and communication devices 160) to basic configuration 102 via bus/interface controller 130. Example output devices 140 include a graphics processing unit 144 and an audio processing unit 146, which may be configured to communicate to various external devices such as a display or speakers via one or more A/V ports 142. Example peripheral interfaces include a serial interface controller 150 or a parallel interface controller 152, which may be configured to communicate with external devices such as input devices (e.g., keyboard, mouse, pen, voice input device, touch input device, etc.) or other peripheral devices (e.g., printer, scanner, etc.) via one or more I/O ports 148. An example communication device 160 includes a network controller 154, which may be arranged to facilitate communications with one or more other computing devices 158 over a network communication link via one or more communication ports 156.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 100 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 100 may also be implemented as a personal computer including both laptop computer and non-laptop computer configurations.

Depending on the desired configuration, system memory 6 may be of any type including but not limited to volatile memory (such as RAM), non-volatile memory (such as ROM, flash memory, etc.) or any combination thereof.

System memory 106 may include an operating system 120, one or more applications 122, and program data 126. Application 122 includes tissue characterization application 108 executed by processor 104. The tissue characterization application 108 is configured to receive measurements from optical data from the spectrometer and processes the optical data using one or more processors. In one embodiment, characteristics of various types of tissues are stored in program data 126. The tissue characterization application may then display the results on a display device.

The above-described techniques provide several advantages including minimizing bleeding while taking measurements with the hollow tool. Since the hollow tool is used along with the stylet during insertion, the orifice is plugged by the stylet and there is reduced disruption of the region of interest. Moreover, since the probe protrudes from the orifice and the tip of the probe is in contact with the region of interest, accurate measurements can be taken enabling more accurate diagnosis of the region of interest.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A sensor system adapted for characterizing a region of interest within a subject, the sensor system comprising:
a hollow tool configured to be inserted into the subject, the hollow tool comprising an orifice;
a probe disposed within the hollow tool and configured to transmit data from the region of interest; wherein the probe protrudes from the orifice of the hollow tool and a tip of the probe is in contact with the region of interest; and
processing circuitry coupled to the probe and configured to process the sensed data to characterize the region of interest.

2. The sensor system of claim 1, wherein the hollow tool comprises:
a cyclindrical hollow tube with a first end and a second end; wherein the orifice is located adjacent to a first end of the cylindrical hollow tube; and
a pointed structure attachable to the first end of the cylindrical hollow tube and configured to facilitate entry into the subject; wherein the pointed structure comprises a sharp component disposed outside hollow tube and a guiding component lodged within the hollow tube.

3. The sensor system of claim 2, wherein the probe is inserted into the hollow tool from the first end and slides out of the orifice.

4. The sensor system of claim 3, wherein the guiding component is formed at an angle with respect to the horizontal axis of the hollow tube.

5. The sensor system of claim 4, wherein the angle is about 10-45 degrees.

6. The sensor system of claim 5, wherein the probe is guided towards the orifice by the guiding component disposed within the hollow tool.

7. The sensor system of claim 1, wherein the hollow tube comprises a first indexing collar configured to indicate an orientation of the orifice.

8. The sensor system of claim 1, wherein the probe comprises a second indexing collar configured to align the probe with the orifice.

9. The sensor system of claim 1, wherein the probe is inserted into the hollow tool after insertion into the subject.

10. The sensor system of claim 1, wherein the probe is embedded within the hollow tool using a bonding adhesive.

11. The sensor system of claim 1, further comprising a stylet be disposed within the hollow tool at the time of insertion into the subject, wherein the stylet configured to minimise bleeding.

12. The sensor system of claim 1, wherein the probe comprises optical sensors configured to sense optical data from the region of interest.

13. The sensor system of claim 1, wherein the probe comprises thermal sensors configured to sense thermal data from the region of interest.

14. A hollow tool adapted for insertion into a subject, the hollow tool comprising
a cylindrical hollow tube with a first end and a second end;
an orifice located adjacent to a first end of the cylindrical hollow tube and disposed at an angle with respect to a horizontal axis of the cylindrical hollow tube,
a pointed structure attachable to the first end of the cylindrical hollow tube and configured to facilitate entry into the subject; and
an indexing collar at a second end of the cylindrical hollow tube and configured to enable a rotary motion of the hollow tool when inserted into the subject.

15. The hollow tool of claim 14, wherein the pointed structure comprises a sharp component disposed outside hollow tube and a guiding component lodged within the hollow tube.

16. The hollow tool of claim 15, wherein the guiding component is formed at angle with respect to the horizontal axis for the probe.

17. The hollow tool of claim 16, wherein the angle ranges from about 10 degrees to about 45 degrees.

18. The hollow tool of claim 14, wherein the cylindrical hollow tube is configured to receive a stylet through the first end when being inserted into the subject to minimize bleeding.

19. The hollow tool of claim 14, wherein the cylindrical hollow tube is configured to receive probe through the first end while the hollow tool is inserted into the subject; wherein the probe is inserted into the hollow tool after the stylet has been removed therefrom.

20. The hollow tool of claim 14, wherein the hollow tube comprises a first indexing collar configured to indicate an orientation of the orifice.
